(19) United States
(12) Patent Application Publication
(10) Pub. No.: US 2026/0272060 A1
(43) Pub. Date: Sep. 17, 2026

(54) HOLDER FOR AN INHALER ARTICLE

(71) Applicant: Philip Morris Products S.A., Neuchatel (CH)

(72) Inventor: Onur DAYIOGLU, Neuchâtel (CH)

(73) Assignee: Philip Morris Products S.A., Neuchatel (CH)

(21) Appl. No.: 19/473,235

(22) PCT Filed: Apr. 9, 2024
(86) PCT No.: PCT/EP2024/059624

§ 371 Date: Oct. 7, 2025

(30) Foreign Application Priority Data
Apr. 14, 2023 (EP) 23167951

(51) Int. Cl. A24F 42/20; A24F 47/00

(52) U.S. Cl. CPC A24F 42/20; A24F 47/00

(57) ABSTRACT

A holder for an inhaler article, including: a housing including a housing cavity; a sleeve within the cavity and including a sleeve cavity to receive the article, the sleeve being movable within the housing cavity between first and second positions; a piercing element to pierce the article within the sleeve when the sleeve is moved from the first to second position; a lockout mechanism including first and second lever members of the sleeve and a retaining section of the housing; and a protruding element at a bottom end of the housing cavity, to protrude into a distal end of the article on insertion of the article into the sleeve cavity, the first and second lever members are in a neutral configuration when the article is not in the sleeve, and are to be moved from the neutral into a working configuration when the article is being inserted into the sleeve.

The present invention relates to a holder for an inhaler article that assists in preventing activation of the inhaler article more than once.

Certain inhaler articles retain capsules containing dry powder. These capsules may be activated by piercing the capsule wall. Typically, the user does not receive an indication that the capsule has been pierced (or “activated”). This may lead to the user activating the capsule multiple times. Activating the capsule more than once may alter the powder delivery by increasing the size of a pierced hole or increasing the number of holes resulting in increased powder delivery and a shortened user experience.

It would be desirable to provide a holder for an inhaler article that may prevent users from accidentally piercing a capsule or activating the inhaler article more than once when the inhaler article is inserted into a sleeve for use.

It would be desirable to provide a holder for an inhaler article that prevents multiple activations of an inhaler article powder capsule received within the holder. It would be desirable to provide an inhaler article system that is convenient to use by a user.

It would be desirable to provide a durable holder for inhaler articles. It would be desirable to provide a holder for inhaler articles with a long product lifetime. It would be desirable to provide a holder for an inhaler article with a mechanism that prevents multiple activations of an inhaler article powder capsule received within the holder, the mechanism having a long life-time and reliably functioning over many use cycles.

According to an embodiment of the present invention there is provided a holder for an inhaler article. The holder may comprise a housing comprising a housing cavity. The holder may comprise a sleeve positioned within the housing cavity. The sleeve may comprise a sleeve cavity arranged to receive the inhaler article. The sleeve may be movable within the housing cavity between a first position and a second position. The holder may comprise a piercing element arranged to pierce the inhaler article received within the sleeve when the sleeve is moved from the first position to the second position. The holder may comprise a lockout mechanism. The lockout mechanism may comprise at least one lever member of the sleeve, preferably the lockout mechanism may comprise at least a first lever member of the sleeve and a second lever member of the sleeve. The lockout mechanism may comprise a retaining section of the housing. The at least one lever member may be arranged to be in a neutral configuration when the inhaler article is not inserted into the sleeve. The at least one lever member may be arranged to be moved, by the inhaler article, from the neutral configuration into a working configuration when the inhaler article is being inserted into the sleeve. When being in the working configuration, the at least one lever member may be arranged to engage with the retaining section as the sleeve moves from the second position back to the first position. The retaining section may be arranged to hold the at least one lever member when being engaged with the retaining section, thus retaining the sleeve in the first position. The at least one lever member may be arranged to disengage from the retaining section when returning into the neutral configuration when the inhaler article is removed from the sleeve.

According to an embodiment of the present invention there is provided a holder for an inhaler article. The holder comprises a housing comprising a housing cavity. The holder comprises a sleeve positioned within the housing cavity. The sleeve comprises a sleeve cavity arranged to receive the inhaler article. The sleeve is movable within the housing cavity between a first position and a second position. The holder comprises a piercing element arranged to pierce the inhaler article received within the sleeve when the sleeve is moved from the first position to the second position. The holder comprises a lockout mechanism. The lockout mechanism comprises at least one lever member of the sleeve. The lockout mechanism comprises a retaining section of the housing. The at least one lever member is arranged to be in a neutral configuration when the inhaler article is not inserted into the sleeve. The at least one lever member is arranged to be moved, by the inhaler article, from the neutral configuration into a working configuration when the inhaler article is being inserted into the sleeve. When being in the working configuration, the at least one lever member is arranged to engage with the retaining section as the sleeve moves from the second position back to the first position. The retaining section is arranged to hold the at least one lever member when being engaged with the retaining section, thus retaining the sleeve in the first position. The at least one lever member is arranged to disengage from the retaining section when returning into the neutral configuration when the inhaler article is removed from the sleeve.

A holder for an inhaler article which may prevent users from accidentally piercing a capsule is provided. A holder for an inhaler article which may prevent users from accidentally activating the inhaler article more than once when the inhaler article is inserted into a sleeve for use is provided.

A holder for an inhaler article that prevents multiple activations of an inhaler article powder capsule received within the holder may be provided. An inhaler article system that is convenient to use by a user may be provided.

A durable holder for inhaler articles may be provided. A holder for inhaler articles with a long product lifetime may be provided. A holder for an inhaler article with a mechanism that prevents multiple activations of an inhaler article powder capsule received within the holder, the mechanism having a long life-time and reliably functioning over many use cycles may be provided. By the holder of the present invention, it may not be necessary to include an additional guide into the lockout mechanism A less complex holder may be provided. A more durable holder may be provided.

The at least one lever member may comprise one lever member. The at least one lever member may comprise a plurality of lever members, for example 2, 3, 4, 5, 6, 7, 8, 9, or 10 lever members. The at least one lever member may comprise at least a first lever member and a second lever member. The at least one lever member may comprise a first lever member and a second lever member.

According to an embodiment of the present invention there is provided a holder for an inhaler article. The holder comprises a housing comprising a housing cavity. The holder comprises a sleeve positioned within the housing cavity. The sleeve comprises a sleeve cavity arranged to receive the inhaler article. The sleeve is movable within the housing cavity between a first position and a second position. The holder comprises a piercing element arranged to pierce the inhaler article received within the sleeve when the sleeve is moved from the first position to the second position. The holder comprises a lockout mechanism. The lockout mechanism comprises a first lever member of the sleeve. The lockout mechanism comprises a second lever member of the sleeve. The lockout mechanism comprises a retaining section of the housing. The first and second lever members are arranged to be in a neutral configuration when the inhaler article is not inserted into the sleeve. The first and second lever members are arranged to be moved, by the inhaler article, from the neutral configuration into a working configuration when the inhaler article is being inserted into the sleeve. When being in the working configuration, the first and second lever members are arranged to engage with the retaining section as the sleeve moves from the second position back to the first position. The retaining section is arranged to hold the first and second lever members when being engaged with the retaining section, thus retaining the sleeve in the first position. The first and second lever members are arranged to disengage from the retaining section when returning into the neutral configuration when the inhaler article is removed from the sleeve.

By the holder of the present invention comprising at least a first lever member and a second lever member, a particularly durable holder for inhaler articles may be provided. By the holder of the present invention comprising at least a first lever member and a second lever member, a holder for inhaler articles with a particularly long product lifetime may be provided. By the holder of the present invention comprising at least a first lever member and a second lever member, a holder for an inhaler article with a mechanism that prevents multiple activations of an inhaler article powder capsule received within the holder, the mechanism having a particularly long life-time and reliably functioning over many use cycles may be provided.

The retaining section may comprise at least one retaining element for engaging with the at least one lever member. The retaining section may comprise a first retaining element for engaging with the first lever member and a second retaining element for engaging with the second lever member. The first and second retaining elements may be arranged at opposing sides of the housing cavity.

The first and second lever members may be arranged at opposing sides of the sleeve cavity.

The holder may comprise at least one spring element for pressing the at least one lever member towards the neutral configuration. The holder may comprise a first spring element for pressing the first lever member towards the neutral configuration. The holder may comprise a second spring element for pressing the second lever member towards the neutral configuration.

The at least one spring element may be blade-shaped. The first and second spring elements may be blade-shaped.

The at least one spring element may be made of a metal or an alloy. The first and second spring elements may be made of a metal or an alloy. The metal or alloy may be a stainless steel. The stainless steel may be an AISI 301 stainless steel.

The at least one spring element may be fixed to the housing. The first and second spring elements may be fixed to the housing.

A first end of the at least one spring element may be fixed to the housing. A first end of the at least one spring element may be fixed to the housing, and a second end of the at least one spring element may be pointing into the housing cavity to press onto the respective at least one lever member of the sleeve positioned within the housing cavity.

Each a first end of the first and second spring elements may be fixed to the housing. Each a first end of the first and second spring elements may be fixed to the housing, and a second end of the first and second spring elements may be pointing into the housing cavity to press onto the respective first and second lever members of the sleeve positioned within the housing cavity.

The at least one spring element may advantageously assure that the at least one lever member returns from the working configuration into the neutral configuration when the inhaler article is removed even after many use cycles. A durable mechanism may be provided.

The sleeve may comprise a polymeric material. the polymeric material may comprise or consist of one or more of polycarbonate, polycarbonate with glass fiber, and POM (polyacetal).

The sleeve may be made of a polymeric material comprising between 5 percent by weight and 50 percent by weight, or between 5 percent by weight and 15 percent by weight, or preferably between 15 percent by weight and 25 percent by weight, or between 25 percent by weight and 35 percent by weight, preferably about 30 percent by weight of a fiber material.

The fiber material may be a glass fiber material. The polymeric material may be a poly carbonate based material and the fiber material may be a glass fiber material.

By including a fiber material into the polymeric material, a sleeve which is less prone to permanent deformation may be provided. By including a fiber material into the polymeric material, a sleeve with at least one lever member which is less prone to permanent deformation may be provided. A more durable holder may be provided. A holder with a lockout mechanism having a long life-time and reliably functioning over many use cycles may be provided.

The holder may be arranged such that the sleeve moves between the first and second positions along a longitudinal direction of the holder. The holder may be arranged such that at least a portion of each of the at least one lever members moves along a transverse direction when changing between the working configuration and the neutral configuration. The holder may be arranged such that at least a portion of each the first and second lever members moves along a transverse direction when changing between the working configuration and the neutral configuration. The transverse direction is substantially perpendicular to the longitudinal direction.

The at least one lever member may extend into the sleeve cavity in the neutral configuration. The first and second lever members may extend into the sleeve cavity in the neutral configuration.

The retaining section may be shaped to hold at least a portion of each of the at least one lever members within the retaining section to resist movement of the sleeve when the at least one lever member reaches the retaining section. The retaining section may be shaped to hold at least a portion of each of the first and second lever members within the retaining section to resist movement of the sleeve when the first and second lever members reach the retaining section.

The retaining section may comprise at least one abutment surface arranged to resist lateral movement of the at least one lever member. The retaining section may comprise at least one abutment surface arranged to resist lateral movement of the first and second lever members.

The at least one lever member may be integrally formed with a tubular body of the sleeve. The first and second lever members may be integrally formed with a tubular body of the sleeve. The first and second lever members and the tubular body of the sleeve may form a monolithic piece. The at least one lever member may be a distinct component attached to a tubular body of the sleeve. The first and second lever members may be distinct components attached to a tubular body of the sleeve.

The at least one lever member may comprise a first end coupled to a tubular body of the sleeve. The at least one lever member may comprise a second end comprising a tip engagement feature extending from the respective lever member.

The at least one lever member may have an elongate shape extending between a first end and a second end. The at least one lever member may have a longitudinal axis in the neutral configuration that is substantially parallel with a longitudinal axis of the sleeve.

Each of the first and second lever members may comprise a first end coupled to a tubular body of the sleeve. The end may be integrally formed with the tubular body of the sleeve. Each of the first and second lever members may comprise a second end comprising a tip engagement feature extending from the respective lever member.

The tip engagement feature may be configured for contacting the inhaler article, when the inhaler article is inserted into the sleeve.

Advantageously, at least one lever member coupled or formed integrally to the sleeve may provide a simple mechanism with few moving parts to prevent accidental piercing of a capsule or inhaler article. Additionally, at least one lever member that is an elongate member may allow lateral biasing and movement of the at least one lever member without requiring any complex additional features of the sleeve.

The at least one lever member may be flexible so that the lever member deflects outwardly away from a longitudinal axis of the sleeve into the working configuration when the inhaler article is received in the sleeve cavity. The at least one lever member may be biased towards the neutral position so that the lever member moves into the neutral position when the inhaler article is removed from the sleeve, thus allowing movement of the sleeve.

The at least one lever member may be elastically deformable or bendable such that the at least one lever member may be bent in a range of about 1.0 millimeter to about 2.0 millimeters outwardly away from the longitudinal axis when an inhaler article is received in the sleeve. Preferably, the at least one lever member may be moved or bent about 1.5 millimeters outwardly.

Each of the first and second lever members may be a lever extending from a first end to a second end, wherein the first end is coupled to the sleeve and the second end comprises a tip engagement feature extending from the lever. In the neutral position, the lever member may be configured such that the second end extends into the sleeve cavity. The lever member may be configured to be in the neutral position when the inhaler article is not within the sleeve cavity. The lever member may be a cantilever.

Each of the first and second lever members may be a cantilever extending from a first end to a second end. The first end may be coupled to the sleeve. The second end may be a free end such that the second end may move independently of the first end. In other words, the second end may be bent or moved in directions different than directions of movement of the first end. The second end may be configured to engage with the inhaler article when the inhaler article is received in the sleeve cavity. The cantilever may be configured to bend or deform from the first end to the second end as it is moved by the inhaler article.

The tip engagement feature may be configured to directly contact the inhaler article and to be moved by the contact with the inhaler article when the inhaler article is being inserted into the sleeve. The tip engagement feature may comprise a protrusion pointing towards a longitudinal center axis of the sleeve cavity.

An angle between a distal end face of the at least one lever member and a plane perpendicular to a longitudinal axis of the at least one lever member may be between 2 degrees and 12 degrees, preferably between 3 degrees and 10 degrees.

The holder may comprise a protruding element. The protruding element may be arranged at a bottom end of the housing cavity. The protruding element may be arranged at a distal end of the housing cavity. The protruding element may be arranged to protrude into a distal end of the inhaler article on insertion of the inhaler article into the sleeve cavity. The protruding element may be arranged to protrude into the sleeve cavity when the sleeve is in the second position. The protruding element may have a tubular shape. An outer diameter of the protruding element may be less than an inner diameter of the sleeve.

The holder may be configured such that, when the sleeve is in the second position, the protruding element is arranged between the tip engagement features of the first and second lever members along a transverse direction. The holder may be configured such that, when the sleeve is in the second position, the protruding element is sandwiched between the tip engagement features of the first and second lever members.

The protruding element may be configured to move together with the sleeve when the sleeve moves between the first and second positions.

The holder may comprise a compressible spring element, for example a helical spring. The compressible spring element may be configured to push the sleeve in a direction towards the first position. The holder may be configured such that the compressible spring element is in a compressed configuration when the sleeve is in the second position. The holder may be configured such that the compressible spring element is in a relaxed configuration when the sleeve is in the second position.

A first end portion of the compressible spring element may be connected to the sleeve. A first end portion of the compressible spring element may be connected to the protruding element. A first end portion of the compressible spring element may be connected to a connector element. The protruding element may be connected to the connector element. One or both of the protruding element and the connector element may be configured to be movable. One or both of the protruding element and the connector element may be configured to move together with the sleeve when the sleeve moves between the first and second positions. A second end of the compressible spring element may be connected to a non-movable part of the housing.

One or both of the protruding element and the connector element may be configured to move relative to the piercing element when the sleeve moves between the first and second positions. The sleeve may be configured to move relative to the piercing element when the sleeve moves between the first and second positions.

The protruding element may comprise an inner channel. The piercing element may be configured to be pushed through the inner channel of the protruding element upon movement of the sleeve from the first position into the second position. The piercing element may be configured to move along the inner channel of the protruding element upon movement of the sleeve from the first position into the second position. The connector element may comprise an inner channel. The piercing element may be configured to be pushed through the inner channel of the connector element upon movement of the sleeve from the first position into the second position. The piercing element may be configured to move along the inner channel of the connector element upon movement of the sleeve from the first position into the second position. The protruding element and the connector element may each comprise an inner channel. The piercing element may be configured to be pushed through the inner channels of the protruding element and the connector element upon movement of the sleeve from the first position into the second position. The piercing element may be configured to move along the inner channels of the protruding element and the connector element upon movement of the sleeve from the first position into the second position.

The piercing element may be configured to pierce the capsule received within the inhaler article. The piercing element may be configured to be pushed through the inner channels of the protruding element and the connector element to pierce the capsule received within the inhaler article. The piercing element may be configured to be pushed through the inner channels of the protruding element and the connector element to pierce the capsule received within the inhaler article upon movement of the sleeve from the first position into the second position.

An inner side of a wall of the housing may comprises a plurality of circumferentially extending rib elements. The rib elements may provide additional mechanical stability to the housing. The rib elements may provide additional rigidity to the housing.

According to an embodiment of the present invention there is provided an inhaler system comprising the holder as described herein and an inhaler article. The inhaler article may comprise a capsule disposed within a capsule cavity of the inhaler article. The capsule may contain pharmaceutically active particles. The pharmaceutically active particles may comprise nicotine.

The inhaler system may be configured to fold a distal end wall of the inhaler article inwardly towards a tubular sidewall of the inhaler article upon movement of the sleeve into the second position when the inhaler article is inserted into the sleeve.

The at least one lever member may be arranged to be moved, by the inhaler article, from the neutral configuration into the working configuration when the inhaler article is being inserted into the sleeve such that the protruding element protrudes into the distal end of the inhaler article. The protruding element may be configured to deform the distal end wall of the inhaler article. The protruding element may be configured to deform the distal end wall of the inhaler article when the inhaler article is inserted into the sleeve. The protruding element may be configured to fold at least a part of the distal end wall of the inhaler article inwardly towards the tubular sidewall of the inhaler article upon movement of the sleeve from the first position into the second position. The protruding element may be configured to fold at least a part of the distal end wall of the inhaler article inwardly towards the tubular sidewall of the inhaler article upon movement of the inhaler article with respect to the protruding element. The protruding element may be configured to fold at least a part of the distal end wall of the inhaler article inwardly towards the tubular sidewall of the inhaler article upon movement of the inhaler article with respect to the protruding element, such that the folded part of the distal end wall of the inhaler article is coaxially arranged between the protruding element and the at least one lever member. The protruding element may be configured to fold at least a part of the distal end wall of the inhaler article inwardly towards the tubular sidewall of the inhaler article upon movement of the inhaler article with respect to the protruding element, such that the folded part of the distal end wall of the inhaler article is coaxially arranged between the protruding element and the tip engagement feature of the at least one lever member. The holder may be configured such that, when the sleeve is in the second position, the folded distal end wall of the inhaler article is sandwiched between the protruding element and the at least one lever member. The holder may be configured such that, when the sleeve is in the second position, the folded distal end wall of the inhaler article is sandwiched between the protruding element and the tip engagement feature of the at least one lever member. By folding the part of the distal end wall of the inhaler article, a thicker overall tubular sidewall of the inhaler article at the distal end of the inhaler article may be provided. By the thicker sidewall, at least one lever member may be pushed in a radially outward direction from the neutral configuration into the working configuration.

The protruding element may be configured to protrude into the distal end of the inhaler article when the sleeve moves from the first position into the second position. The protruding element may be configured to protrude into the distal end of the inhaler article such that the protruding element moves together with the sleeve when the sleeve moves from the second position back into the first position.

Each of the at least one lever members may have a longitudinal axis in a neutral position that is parallel with a longitudinal axis of the sleeve. The sleeve may be configured to receive and be disposed about a distal end region of an inhaler article. The sleeve may have a base surface for contacting the distal end of an inhaler article. The base surface may define a distal end of the sleeve and oppose an open proximal end of the sleeve. The sleeve may extend from the distal end to the open proximal end. The sleeve may retain an inhaler article via interference fit. The sleeve may include an aperture at the distal end of the sleeve and the piercing element may extend thorough the aperture. The distal end of the inhaler article may contact the base surface of the sleeve and urge the sleeve to travel toward the piercing element. The sleeve may be co-axial with the piercing element. The sleeve may align the inhaler article so that the piercing element reliably activates capsule within the inhaler article. The sleeve may also mechanically hold the piercing element and support the piercing element to prevent or mitigate deflection of the piercing element.

The sleeve may define a first air inlet zone comprising at least one air aperture through the sleeve. The first air inlet zone may include two or more, three or more, four or more, or from about 1 to about 10 air apertures, or from about 3 to about 9 air apertures. The first air inlet zone is proximate to a proximal end of the sleeve. The first air inlet zone is configured to allow air to flow from an inside of the sleeve to an airflow channel formed between the sleeve and the housing inner surface.

The sleeve may comprise a second air inlet zone comprising at least one air aperture through the sleeve. The second air inlet zone may include two or more, three or more, four or more, or from about 1 to about 10 air apertures, or from about 3 to about 9 air apertures. The second air inlet zone is proximate to a distal end of the sleeve. The second air inlet zone is configured to allow air to flow from the airflow channel to an inside of the sleeve.

In some embodiments, the portion of the inner cavity between the first air inlet zone and the second air inlet zone may have a reduced diameter. In these embodiments, the air (inhalation air) is unable to pass from the proximal end of the sleeve to the distal end of the sleeve between the sleeve and the inhalation article. In these embodiments, the provision of a first and second air inlet zone advantageously allows air to enter the open end of the holder and pass to the distal end of an inhaler article while still allowing the article to be securely retained in the sleeve. Thus, the inhaler article may be consumed by a user while the inhaler article is contained within the holder.

The holder may include a retaining ring element fixed to the open proximal end of the housing. The retaining ring element may retain the sleeve within the inhaler article cavity. The retaining ring may have a thickness sufficient to stop or retain the movement of the sleeve within the housing cavity of the holder.

The holder may include a compressible spring element configured to bias the sleeve between a relaxed (first) and compressed (second) position towards the open proximal end of the housing. The compressible spring element may be contained within the inhaler article cavity of the holder and be compressed as the movable sleeve and inhaler article move toward the piercing element. The compressible spring element may be arranged between the sleeve and the distal end of the housing and may contact the sleeve and distal end of the housing. The compressible spring element may be disposed about the piercing element. The compressible spring element may be co-axial with the piercing element. The compressible spring element may be a conical spring.

The compressible spring element may be fixed to the distal end of the holder. The compressible spring element may be fixed to the distal end of the sleeve. The compressible spring element may be fixed to the distal end of the protruding element. The compressible spring element may be fixed to the distal end of the connector element. The compressible spring element may be fixed to both the distal end of the holder and the distal end of the sleeve. The compressible spring element may be fixed to both the distal end of the holder and the distal end of the protruding element. The compressible spring element may be fixed to both the distal end of the holder and the distal end of the connector element.

The compressible spring element may be a conical spring. The conical spring advantageously may provide a low-profile design so that it may provide a more flexible design and smaller overall compression thickness. The provision of a conical spring may also advantageously reduce the likelihood that the spring will buckle when compressed compared to a cylindrical spring.

The compressible spring element may bias the inhaler article out of engagement with and away from the piercing element once the piercing element activates the inhaler article. The compressible spring element may be disposed about the piercing element. The compressible spring element may be coaxial with the piercing element. The piercing element may extend beyond the compressible spring element when the compressible spring element is in a relaxed position. The piercing element may extend beyond the compressible spring element when the compressible spring element is in a compressed position. The piercing element may extend beyond the compressible spring element when the compressible spring element is in both the relaxed position and the compressed position. The piercing element may extend beyond the compressible spring element when the sleeve compresses the compressible spring element.

Recessing the piercing element into the housing may protect the piercing element from coming into contact with surfaces not intended to be received within the piercing element. Recessing the piercing element into the housing may also protect the piercing element from being damaged or modified by surfaces not intended to be received within the piercing element.

The piercing element length may be any suitable length relative to the housing length. For example, the piercing element length may be about 25% to about 60%, or about 30% to about 50%, of the housing length. A distal end of the piercing element may be fixed to the distal end adjacent to or at the distal end of the housing. A distal end of the piercing element may be fixed to the distal end of the housing at a position adjacent to the distal end of the housing, or at the distal end of the housing. The distal end of the piercing element may be fixed to the distal end of the housing or to a position adjacent to the distal end of the housing.

The piercing element entire length may be coextensive within the housing length.

The piercing element is formed of a rigid material. The rigid material is sufficiently rigid to pierce, puncture or activate a capsule contained within the inhaler article. The piercing element may be formed of a metal. The piercing element may be formed of stainless steel, such as 316 stainless steel, for example. The piercing element may be formed of a polymeric material. The piercing element may be formed of a fibre-reinforced polymeric material.

The housing may be formed of any rigid material. The housing may be formed of a polymeric material. Polymeric materials useful for forming the housing include polycarbonate, polypropylene, polyethylene, nylon, acrylonitrile butadiene styrene, styrene acrylonitrile, polyacrylate, polystyrene, PBT polyester, PET polyester, polyoxymethylene, polysulfone, polyethersulfone, polyethereetherketone, or liquid crystal polymer, for example. Polyproplyene, polyethylene or co-polymers thereof are preferred materials for forming the housing.

The inhaler article may be received into the holder such that the inhaler article outer surface and the holder housing outer surface are concentric. The piercing element longitudinal axis may be coaxial with the housing longitudinal axis, and the inhaler longitudinal axis, when the inhaler article is received within the holder. At least about 50%, or at least about 75% of the housing length may be coextensive with the inhaler length, when the inhaler article is received within the holder.

The holder may be formed by insertion moulding techniques. The piercing element may first be formed by moulding, for example, and then the housing may be moulded around the piercing element bonding to the piercing element. The piercing element may be a metal piercing element, the housing may be moulded around the metal piercing element fixing the metal piercing element to the housing. A metal piercing element may include protrusions or recesses at the distal end of the piercing element to increase surface area of the distal end of the piercing element and improve fixation within the housing moulded material.

An inhaler article air channel may extend through an end cap or endpiece element of the inhaler article to provide airflow through the inhaler article. The air channel supplying airflow to the capsule cavity may be configured to receive or induce a swirling air flow pattern within the capsule cavity of the inhaler body. The air channel configuration may induce rotational air flow or swirling air flow as the air flows through the air channel and through the capsule cavity. Air flow through the inhaler article may enter the inhaler article at the distal end of the inhaler article and moves along the longitudinal axis of the inhaler article to the mouthpiece end. Airflow through the inhaler article may enter the inhaler article along the inhaler body upstream or along the capsule cavity and move along the longitudinal axis of the inhaler article to the mouthpiece end.

The inhaler body may resemble a smoking article or cigarette in size and shape. The inhaler body may have an elongated body extending along the longitudinal axis of the inhaler article. The inhaler body may have a substantially uniform outer diameter along the length of the elongated body. The inhaler body may have a circular cross-section that may be uniform along the length of the elongated body. The inhaler body may have an outer diameter in a range from about 6 mm to about 10 mm, or from about 7 mm to about 10 mm, or about 7 mm to about 9 mm, or about 7 mm to about 8 mm or about 7.2 mm. The inhaler body may have a length (along the longitudinal axis) in a range from about 40 mm to about 80 mm, or from about 40 mm to about 70 mm, or about 40 mm to about 50 mm, or about 45 mm.

The inhaler article may have an open distal end or upstream-most end defined by a tubular element defining a central passage. The open central passage may define a cylindrical open aperture extending from the capsule cavity to the open distal end or upstream-most end of the inhaler article. The open tubular element defining an open central passage may have a length in a range from about 3 mm to about 12 mm, or from about 3 mm to about 7 mm or about 4 mm to about 6 mm, or about 5 mm.

The open tubular element defining an open central passage may be formed of a cellulose material. The open tubular element defining an open central passage may be formed of a cellulose acetate material. Preferably the open tubular element is formed of a biodegradable material. The open tubular element defining an open central passage may have a thickness in a range from about 0.5 mm to about 1.5 mm, or about 0.5 mm to about 1 mm.

The filter element located downstream of the capsule cavity may extend from the capsule cavity to the mouthpiece end of the inhaler article. The filter element may have a length in a range from about 10 mm to about 30 mm, preferably from about 15 mm to about 25 mm and more preferably from about 20 mm to about 22 mm.

The capsule cavity may define a cylindrical space configured to contain a capsule (the capsule may have an obround shape or a circular cross-section, for example). The capsule cavity may have a substantially uniform or uniform diameter along the length of the capsule cavity. The capsule cavity may have a fixed cavity length. The capsule cavity has a cavity inner diameter, orthogonal to the longitudinal axis, and the capsule has a capsule outer diameter. The capsule cavity may be sized to contain an obround capsule. The capsule cavity may have a substantially cylindrical or cylindrical cross-section along the length of the capsule cavity. The capsule cavity may have a uniform inner diameter. The capsule may have an outer diameter that is about 80% to about 95% of the inner diameter of the capsule cavity. The configuration of the capsule cavity relative to the capsule may promote limited movement of the capsule during activation or piercing of the capsule. The capsule cavity may be defined by an open tubular element. The open tubular element may be joined between and in abutting alignment with the open tubular element forming the distal end of the inhaler article and the filter element. These elements may be joined with a wrapper. The open tubular element defining the capsule cavity may be formed of a biodegradable material, such as cardboard or paperboard.

The configuration of the capsule cavity relative to the capsule may promote the capsule to rotate with stability within the capsule cavity. The longitudinal axis of the capsule may rotate with stability co-axially with the longitudinal axis of the inhaler body during inhalation. The configuration of the capsule cavity relative to the capsule may promote the capsule to rotate with some shaking within the capsule cavity.

Stable rotation refers to the longitudinal axis of the inhaler body being substantially parallel or co-axial with the axis of rotation of the capsule. Stable rotation may refer to the absence of procession of the rotating capsule. Preferably the longitudinal axis of the inhaler body may be substantially coextensive with the axis of rotation of the capsule. Stable rotation of the capsule may provide a uniform entrainment of a portion of nicotine particles from the capsule over two or more, or five or more, or ten or more “puffs” or inhalations by a user.

The capsule may be sealed within the inhaler article prior to consumption. The inhaler article may be contained within a sealed or airtight container or bag. The inhaler article may include one or more peelable or removable seal layers to cover the one or more air inlet channels or the air outlet or mouthpiece of the inhaler article.

The capsule may rotate about its longitudinal or central axis when air flows through the inhaler article. The capsule may be formed of an airtight material that may be pierced or punctured by a piercing element that may be separate or combined with the inhaler. The capsule may be formed of a metallic or polymeric material that serves to keep contaminates out of the capsule but may be pierced or punctured by a piercing element prior to consumption of the nicotine particles within the capsule. The capsule may be formed of a polymer material. The polymer material may be hydroxypropylmethylcellulose (HPMC). The capsule may be a size 1 to size 4 capsule, or a size 3 capsule.

The capsule may be nicotine-free. The capsule may comprise nicotine.

The capsule may contain nicotine particles comprising nicotine (also referred to as nicotine powder“ or ”nicotine particles“) and optionally particles comprising flavour (also referred to as ”flavour particles). The capsule may contain a predetermined amount of nicotine particles and optional flavour particles. The capsule may contain enough nicotine particles to provide at least 2 inhalations or “puffs”, or at least about 5 inhalations or “puffs”, or at least about 10 inhalations or “puffs”. The capsule may contain enough nicotine particles to provide from about 5 to about 50 inhalations or “puffs”, or from about 10 to about 30 inhalations or “puffs”. Each inhalation or “puff” may deliver from about 0.1 mg to about 3 mg of nicotine particles to the lungs of the user or from about 0.2 mg to about 2 mg of nicotine particles to the lungs of the user or about 1 mg of nicotine particles to the lungs of the user.

The nicotine particles may have any useful concentration of nicotine based on the particular formulation employed. The nicotine particles may have at least about 1% wt nicotine up to about 30% wt nicotine, or from about 2% wt to about 25% wt nicotine, or from about 3% wt to about 20% wt nicotine, or from about 4% wt to about 15% wt nicotine, or from about 5% wt to about 13% wt nicotine. Preferably, about 50 to about 150 micrograms of nicotine may be delivered to the lungs of the user with each inhalation or “puff”.

The capsule may hold or contain at least about 5 mg of nicotine particles or at least about 10 mg of nicotine particles. The capsule may hold or contain less than about 900 mg of nicotine particles, or less than about 300 mg of nicotine particles, or less than 150 mg of nicotine particles. The capsule may hold or contain from about 5 mg to about 300 mg of nicotine particles or from about 10 mg to about 200 mg of nicotine particles.

When flavour particles are blended or combined with the nicotine particles within the capsule, the flavour particles may be present in an amount that provides the desired flavour to each inhalation or “puff” delivered to the user.

The nicotine particles may have any useful size distribution for inhalation delivery preferentially into the lungs of a user. The capsule may include particles other than the nicotine particles. The nicotine particles and the other particles may form a powder system.

The capsule may hold or contain at least about 5 mg of a dry powder (also referred to as a powder system) or at least about 10 mg of a dry powder. The capsule may hold or contain less than about 900 mg of a dry powder, or less than about 300 mg of a dry powder, or less than about 150 mg of a dry powder. The capsule may hold or contain from about 5 mg to about 300 mg of a dry powder, or from about 10 mg to about 200 mg of a dry powder, or from about 25 mg to about 100 mg of a dry powder. The dry powder or powder system may have at least about 40%, or at least about 60%, or at least about 80%, by weight of the powder system comprised in nicotine particles having a particle size of about 5 micrometers or less, or in a range from about 1 micrometer to about 5 micrometers. The particles comprising nicotine may have a mass median aerodynamic diameter of about 5 micrometers or less, or in a range from about 0.5 micrometers to about 4 micrometers, or in a range from about 1 micrometer to about 3 micrometers or in a range from about 1.5 micrometers to about 2.5 micrometers. The mass median aerodynamic diameter is preferably measured with a cascade impactor. The particles comprising flavour may have a mass median aerodynamic diameter of about

20 micrometers or greater, or about 50 micrometers or greater, or in a range from about 50 to about 200 micrometers, or from about 50 to about 150 micrometers. The mass median aerodynamic diameter is preferably measured with a cascade impactor.

The dry powder may have a mean diameter of about 60 micrometers or less, or in a range from about 1 micrometer to about 40 micrometers, or in a range from about 1.5 micrometers to about 25 micrometers. The mean diameter refers to the mean diameter per mass and is preferably measured by laser diffraction, laser diffusion or an electronic microscope.

Nicotine in the powder system or nicotine particles may be a pharmaceutically acceptable free-base nicotine, or nicotine salt or nicotine salt hydrate. Useful nicotine salts or nicotine salt hydrates include nicotine pyruvate, nicotine citrate, nicotine aspartate, nicotine lactate, nicotine bitartrate, nicotine salicylate, nicotine fumarate, nicotine mono-pyruvate, nicotine glutamate or nicotine hydrochloride, for example. The compound combining with nicotine to form the salt or salt hydrate may be chosen based on its expected pharmacological effect.

The nicotine particles preferably include an amino acid. Preferably the amino acid may be leucine such as L-leucine. Providing an amino acid such as L-leucine with the particles comprising nicotine, may reduce adhesion forces of the particles comprising nicotine and may reduce attraction between nicotine particles and thus reduce agglomeration of nicotine particles. Similarly, adhesion forces to particles comprising flavour may also be reduced thus agglomeration of nicotine particles with flavour particles is also reduced. The powder system described herein thus may be a free-flowing material and possess a stable relative particle size of each powder component even when the nicotine particles and the flavour particles are combined. Preferably, the nicotine may be a surface modified nicotine salt where the nicotine salt particle comprises a coated or composite particle. A preferred coating or composite material may be L-leucine. One particularly useful nicotine particle may be nicotine bitartrate with L-leucine.

The powder system may include a population of flavour particles. The flavour particles may have any useful size distribution for inhalation delivery selectively into the mouth or buccal cavity of a user.

The powder system may have at least about 40%, or at least about 60%, or at least about 80%, by weight of the population of flavour particles of the powder system comprised in particles having a particle size of about 20 micrometers or greater. The powder system may have at least about 40% or at least about 60%, or at least about 80%, by weight of the population of flavour particles of the powder system comprised in particles having a particle size of about 50 micrometers or greater. The powder system may have at least about 40% or at least about 60%, or at least about 80%, by weight of the population of flavour particles of the powder system comprised in particles having a particle size in a range from about 50 micrometer to about 150 micrometers.

The particles comprising flavour may include a compound to reduce adhesion forces or surface energy and resulting agglomeration. The flavour particle may be surface modified with an adhesion reducing compound to form a coated flavour particle. One preferred adhesion reducing compound may be magnesium stearate. Providing an adhesion reducing compound such as magnesium stearate with the flavour particle, especially coating the flavour particle, may reduce adhesion forces of the particles comprising flavour and may reduce attraction between flavour particles and thus reduce agglomeration of flavour particles. Thus, agglomeration of flavour particles with nicotine particles may also be reduced. The powder system described herein thus may possess a stable relative particle size of the particles comprising nicotine and the particles comprising flavour even when the nicotine particles and the flavour particles are combined. The powder system preferably may be free flowing.

Conventional formulations for dry powder inhalation contain carrier particles that serve to increase the fluidization of the active particles since the active particles may be too small to be influenced by simple airflow though the inhaler. The powder system may comprise carrier particles. These carrier particles may be a saccharide such as lactose or mannitol that may have a particle size greater than about 50 micrometers. The carrier particles may be utilized to improve dose uniformity by acting as a diluent or bulking agent in a formulation. The powder system utilized with the nicotine powder delivery system described herein may be carrier-free or substantially free of a saccharide such as lactose or mannitol. Being carrier-free or substantially free of a saccharide such as lactose or mannitol may allow the nicotine and to be inhaled and delivered to the user's lungs at inhalation or airflow rates that are similar to typical smoking regime inhalation or airflow rates.

The nicotine particles and a flavour may be combined in a single capsule. As described above, the nicotine particles and a flavour may each have reduced adhesion forces that result in a stable particle formulation where the particle size of each component does not substantially change when combined. Alternatively, the powder system includes nicotine particles contained within a single capsule and the flavour particles contained within a second capsule.

The nicotine particles and flavour particles may be combined in any useful relative amount so that the flavour particles are detected by the user when consumed with the nicotine particles. Preferably the nicotine particles and a flavour particles form at least about 90% wt or at least about 95% wt or at least about 99% wt or 100% wt of the total weight of the powder system. The inhaler and inhaler system may be less complex and have a simplified airflow path as compared to conventional dry powder inhalers. Advantageously, rotation of the capsule within the inhaler body aerosolizes the nicotine particles or powder system and may assist in maintaining a free-flowing powder. Thus, the inhaler article may not require the elevated inhalation rates typically utilized by conventional inhalers to deliver the nicotine particles described above deep into the lungs.

The inhaler article may use a flow rate of less than about 5 L/min or less than about 3 L/min or less than about 2 L/min or about 1.6 L/min. Preferably, the flow rate may be in a range from about 1 L/min to about 3 L/min or from about 1.5 L/min to about 2.5 L/min. Preferably, the inhalation rate or flow rate may be similar to that of Health Canada smoking regime, that is, about 1.6 L/min.

The inhaler system may be used by a user like smoking a conventional cigarette or vaping an electronic cigarette. Such smoking or vaping may be characterized by two steps: a first step during which a small volume containing the full amount of nicotine desired by the user is drawn into the mouth cavity, followed by a second step during which this small volume comprising the aerosol comprising the desired amount of nicotine is further diluted by fresh air and drawn deeper into the lungs. Both steps are controlled by the user. During the first inhalation step the user may determine the amount of nicotine to be inhaled. During the second step, the user may determine the volume for diluting the first volume to be drawn deeper into the lungs, maximizing the concentration of active agent delivered to the airway epithelial surface. This smoking mechanism is sometimes called “puff-inhale-exhale”.

The pharmaceutically active particles may have a mass median aerodynamic diameter of about 5 micrometers or less, or in a range from about 0.5 micrometers to about 4 micrometers, or in a range from about 1 micrometer to about 3 micrometers.

The capsule may further contain a second population of flavour particles having a mass median aerodynamic diameter of about 20 micrometers or greater, or about 50 micrometers or greater, or in a range from about 50 to about 200 micrometers, or from about 50 to about 150 micrometers.

The term “nicotine” refers to nicotine and nicotine derivatives such as free-base nicotine, nicotine salts and the like.

The term “flavourant” or “flavour” refers to organoleptic compounds, compositions, or materials that alter and are intended to alter the taste or aroma characteristics of nicotine during consumption or inhalation thereof.

The terms “upstream” and “downstream” refer to relative positions of elements of the holder, inhaler article and inhaler systems described in relation to the direction of inhalation air flow as it is drawn through the body of the holder, inhaler article and inhaler systems.

The terms “proximal” and “distal” are used to describe the relative positions of components, or portions of components, of the holder, inhaler article, or system. Holders or elements (such as the sleeve) forming the holder, according to the invention have a proximal end which, in use, receives an inhaler article and an opposing distal end which may be a closed end, or have an end closer to the proximal end of the holder. Inhaler articles, according to the invention have a proximal end. In use, the nicotine particles exit the proximal end of the inhaler article for delivery to a user. The inhaler article has a distal end opposing the proximal end. The proximal end of the inhaler article may also be referred to as the mouth end.

The holder for an inhaler article described herein may be combined with an inhaler article containing a capsule for activating the inhaler article by piercing the capsule, providing reliable activation of the capsule (by puncturing the capsule with the piercing element of the holder) within inhaler article, and releasing the particles contained inside the capsule and enabling the article to deliver the particles to a user. The holder is separate from the inhaler article, but the user may utilize both the inhaler article and the holder while consuming the particles released within the inhaler article. A plurality of these inhaler articles may be combined with a holder to form a system or kit. A single holder may be utilized on 10 or more, or 25 or more, or 50 or more, or 100 or more, inhaler articles to activate (puncture or pierce) a capsule contained within each inhaler article and provide reliable activation and optionally, a visual indication (marking), for each inhaler article of the activation of the inhaler article.

The holder for an inhaler article described herein may be combined with an inhaler article containing a capsule for activating the inhaler article by piercing the capsule, providing reliable activation of the capsule (by puncturing the capsule with the piercing element of the holder) within inhaler article, and releasing the particles contained inside the capsule and enabling the article to deliver the particles to a user. The holder is separate from the inhaler article, but the user may utilize both the inhaler article and the holder while consuming the particles released within the inhaler article. A plurality of these inhaler articles may be combined with a holder to form a system or kit. A single holder may be utilized on 10 or more, or 25 or more, or 50 or more, or 100 or more, inhaler articles to activate (puncture or pierce) a capsule contained within each inhaler article and provide reliable activation and optionally, a visual indication (marking), for each inhaler article of the activation of the inhaler article.

An inhaler system comprises an inhaler article and a holder for an inhaler article as described herein. The sleeve of the holder retains the inhaler article received in the sleeve cavity. A capsule may be disposed within the inhaler article body. An inhaler article may comprise a body extending along an inhaler longitudinal axis from a mouthpiece end to a distal end. A capsule cavity may be defined within the body bounded downstream by a filter element and bounded upstream by a tubular element defining a central passage. The central passage may form an air-inlet aperture extending from the distal end of the body towards the capsule cavity. A capsule may be disposed within the capsule cavity, wherein the central passage may have a smaller diameter then the capsule. Thus, the capsule may not pass through the central passage and is retained within the capsule cavity.

The at least one lever member may be formed of elastically deformable material. Suitable elastically deformable materials may include thermoplastic material. Suitable elastically deformable materials may include polyetheretherketone, polyamide, acetal (polyactal or polyoxymethlene), polybutylene terephthalate, styrene acrylonitrile, or other materials that can be biased and elastically deformed. The at least one lever member may be formed of acetal (polyactal or polyoxymethlene). The at least one lever member may be formed of polyetheretherketone.

Below, there is provided a non-exhaustive list of non-limiting examples. Any one or more of the features of these examples may be combined with any one or more features of another example, embodiment, or aspect described herein.

Example E1: A holder for an inhaler article, the holder comprising:
a housing comprising a housing cavity;
a sleeve positioned within the housing cavity, wherein the sleeve comprises a sleeve cavity arranged to receive the inhaler article and the sleeve is movable within the housing cavity between a first position and a second position;
a piercing element arranged to pierce the inhaler article received within the sleeve when the sleeve is moved from the first position to the second position; and
a lockout mechanism comprising at least one lever member of the sleeve and a retaining section of the housing;
wherein the at least one lever member is arranged to be in a neutral configuration when the inhaler article is not inserted into the sleeve,
wherein the at least one lever member is arranged to be moved, by the inhaler article, from the neutral configuration into a working configuration when the inhaler article is being inserted into the sleeve,
wherein, when being in the working configuration, the at least one lever member is arranged to engage with the retaining section as the sleeve moves from the second position back to the first position,
wherein the retaining section is arranged to hold the at least one lever member when being engaged with the retaining section, thus retaining the sleeve in the first position, and
wherein the at least one lever member is arranged to disengage from the retaining section when returning into the neutral configuration when the inhaler article is removed from the sleeve.

Example E2: The holder according to Example E1, wherein the retaining section comprises at least one retaining element for engaging with the at least one lever member.

Example E3: The holder according to any of the preceding examples, comprising at least one spring element for pressing the at least one lever member towards the neutral configuration.

Example E4: The holder according to Example E3, wherein the at least one spring element is blade-shaped.

Example E5: The holder according to Example E3 or Example E4, wherein the at least one spring element is made of a metal or an alloy, preferably a stainless steel, more preferably an AISI 301 stainless steel.

Example E6: The holder according to any of Examples E3 to E5, wherein the at least one spring element is fixed to the housing.

Example E7: The holder according to Example E6, wherein each a first end of the at least one spring element is fixed to the housing, and each a second end of the at least one spring element is pointing into the housing cavity to press onto the respective at least one lever member of the sleeve positioned within the housing cavity.

Example E8: The holder according to any of the preceding examples, wherein the holder is arranged such that the sleeve moves between the first and second positions along a longitudinal direction of the holder, and wherein the holder is arranged such that at least a portion of each of the at least one lever members moves along a transverse direction when changing between the working configuration and the neutral configuration, the transverse direction being substantially perpendicular to the longitudinal direction.

Example E9: The holder according to any of the preceding examples, wherein the at least one lever member extends into the sleeve cavity in the neutral configuration.

Example E10: The holder according to any of the preceding examples, wherein the retaining section is shaped to hold at least a portion of each of the at least one lever members within the retaining section to resist movement of the sleeve when the at least one lever member reaches the retaining section.

Example E11: The holder according to Example E10, wherein the retaining section comprises at least one abutment surface arranged to resist lateral movement of the at least one lever member.

Example E12: The holder according to any of the preceding examples, wherein the at least one lever member is integrally formed with a tubular body of the sleeve, or
wherein the at least one lever member is a distinct component attached to a tubular body of the sleeve.

Example E13: The holder according to any of the preceding examples, wherein the at least one lever member comprises a first end coupled to a tubular body of the sleeve and a second end comprising a tip engagement feature extending from the respective lever member. Example E14: The holder according to Example E13, wherein the tip engagement feature is configured to directly contact the inhaler article and to be moved by the contact with the inhaler article when the inhaler article is being inserted into the sleeve.

Example E15: The holder according to Example E14, wherein the tip engagement feature comprises a protrusion pointing towards a longitudinal center axis of the sleeve cavity.

Example E16: The holder according to any of the preceding examples, wherein an angle between a distal end face of the at least one lever member and a plane perpendicular to a longitudinal axis of the at least one lever member is between 2 degrees and 12 degrees, preferably between 3 degrees and 10 degrees.

Example E17: A holder for an inhaler article, the holder comprising:
a housing comprising a housing cavity;
a sleeve positioned within the housing cavity, wherein the sleeve comprises a sleeve cavity arranged to receive the inhaler article and the sleeve is movable within the housing cavity between a first position and a second position;
a piercing element arranged to pierce the inhaler article received within the sleeve when the sleeve is moved from the first position to the second position; and
a lockout mechanism comprising a first lever member of the sleeve, a second lever member of the sleeve, and a retaining section of the housing;
wherein the first and second lever members are arranged to be in a neutral configuration when the inhaler article is not inserted into the sleeve,
wherein the first and second lever members are arranged to be moved, by the inhaler article, from the neutral configuration into a working configuration when the inhaler article is being inserted into the sleeve,
wherein, when being in the working configuration, the first and second lever members are arranged to engage with the retaining section as the sleeve moves from the second position back to the first position,
wherein the retaining section is arranged to hold the first and second lever members when being engaged with the retaining section, thus retaining the sleeve in the first position, and
wherein the first and second lever members are arranged to disengage from the retaining section when returning into the neutral configuration when the inhaler article is removed from the sleeve.

Example E18: The holder according to Example E17, wherein the retaining section comprises a first retaining element for engaging with the first lever member and a second retaining element for engaging with the second lever member.

Example E19: The holder according to Example E17 or Example E18, wherein the first and second lever members are arranged at opposing sides of the sleeve cavity.

Example E20: The holder according to any of Examples E17 to E19, comprising a first spring element for pressing the first lever member towards the neutral configuration, and a second spring element for pressing the second lever member towards the neutral configuration.

Example E21: The holder according to Example E20, wherein the first and second spring elements are blade-shaped.

Example E22: The holder according to Example E20 or Example E21, wherein the first and second spring elements are made of a metal or an alloy, preferably a stainless steel, more preferably an AISI 301 stainless steel.

Example E23: The holder according to any of Examples E20 to E22, wherein the first and second spring elements are fixed to the housing.

Example E24: The holder according to Example E23, wherein each a first end of the first and second spring elements is fixed to the housing, and each a second end of the first and second spring elements is pointing into the housing cavity to press onto the respective first and second lever members of the sleeve positioned within the housing cavity.

Example E25: The holder according to any of the preceding examples, wherein the sleeve comprises a polymeric material, preferably one or more of: polycarbonate, polycarbonate with glass fiber, and POM (polyacetal).

Example E26: The holder according to Example E25, wherein the sleeve is made of a polymeric material comprising between 5 percent by weight and 50 percent by weight, or between 5 percent by weight and 15 percent by weight, or preferably between 15 percent by weight and 25 percent by weight, or between 25 percent by weight and 35 percent by weight, preferably about 30 percent by weight of a fiber material.

Example E27: The holder according to Example E26, wherein the fiber material is a glass fiber material, preferably, wherein the polymeric material is a poly carbonate based material and the fiber material is a glass fiber material.

Example E28: The holder according to any of Examples E17 to E27, wherein the holder is arranged such that the sleeve moves between the first and second positions along a longitudinal direction of the holder, and wherein the holder is arranged such that at least a portion of each the first and second lever members moves along a transverse direction when changing between the working configuration and the neutral configuration, the transverse direction being substantially perpendicular to the longitudinal direction.

Example E29: The holder according to any of Examples E17 to E28, wherein the first and second lever members extend into the sleeve cavity in the neutral configuration.

Example E30: The holder according to any of Examples E17 to E29, wherein the retaining section is shaped to hold at least a portion of each of the first and second lever members within the retaining section to resist movement of the sleeve when the first and second lever members reach the retaining section.

Example E31: The holder according to Example E30, wherein the retaining section comprises at least one abutment surface arranged to resist lateral movement of the first and second lever members.

Example E32: The holder according to any of Examples E17 to E31, wherein the first and second lever members are integrally formed with a tubular body of the sleeve, or wherein the first and second lever members are distinct components attached to a tubular body of the sleeve.

Example E33: The holder according to any of Examples E17 to E32, wherein each of the first and second lever members comprises a first end coupled to a tubular body of the sleeve and a second end comprising a tip engagement feature extending from the respective lever member.

Example E34: The holder according to Example E33, wherein the tip engagement feature is configured to directly contact the inhaler article and to be moved by the contact with the inhaler article when the inhaler article is being inserted into the sleeve.

Example E35: The holder according to Example E34, wherein the tip engagement feature comprises a protrusion pointing towards a longitudinal center axis of the sleeve cavity.

Example E36: The holder according to any of Examples E17 to E35, wherein an angle between a distal end face of the first lever member and a plane perpendicular to a longitudinal axis of the first lever member is between 3 degrees and 10 degrees, and wherein an angle between a distal end face of the second lever member and a plane perpendicular to a longitudinal axis of the second lever member is between 3 degrees and 10 degrees.

Example E37: The holder according to any of the preceding examples, comprising a protruding element arranged at a bottom end of the housing cavity, the protruding element being arranged to protrude into a distal end of the inhaler article on insertion of the inhaler article into the sleeve cavity, optionally wherein the protruding element is arranged to protrude into the sleeve cavity when the sleeve is in the second position.

Example E38: The holder according to Example E37, wherein the protruding element has a tubular shape.

Example E39: The holder according to any of the preceding examples, wherein an inner side of a wall of the housing comprises a plurality of circumferentially extending rib elements.

Example E40: An inhaler system comprising the holder according to any one of the preceding examples and an inhaler article, the inhaler article comprising a capsule disposed within a capsule cavity of the inhaler article, preferably wherein the capsule contains pharmaceutically active particles, optionally wherein the pharmaceutically active particles comprise nicotine.

Example E41: The inhaler system according to Example E40, wherein the system is configured to fold a distal end wall of the inhaler article inwardly towards a tubular sidewall of the inhaler article upon movement of the sleeve into the second position when the inhaler article is inserted into the sleeve.

Features described in relation to one embodiment may equally be applied to other embodiments of the invention.

The invention will be further described, by way of example only, with reference to the accompanying drawings in which:

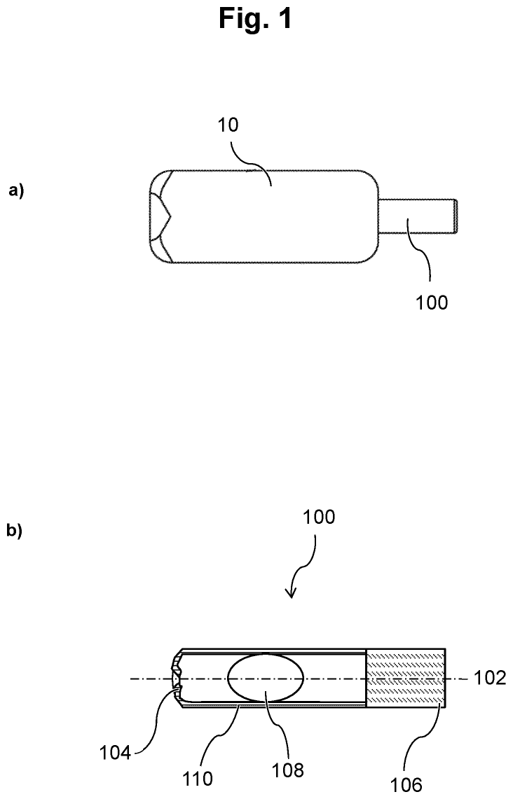

FIG. 1a shows an inhaler system;

FIG. 1b shows an inhaler article;

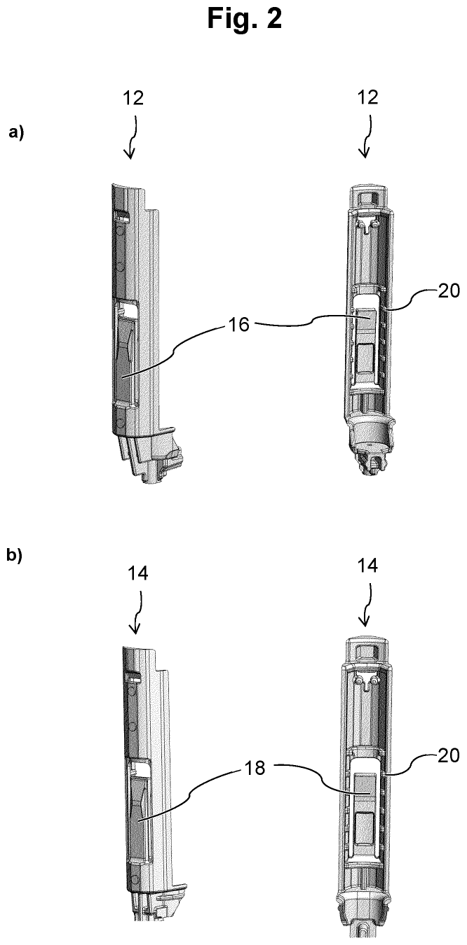

FIGS. 2a and 2b show a housing of a holder for an inhaler article;

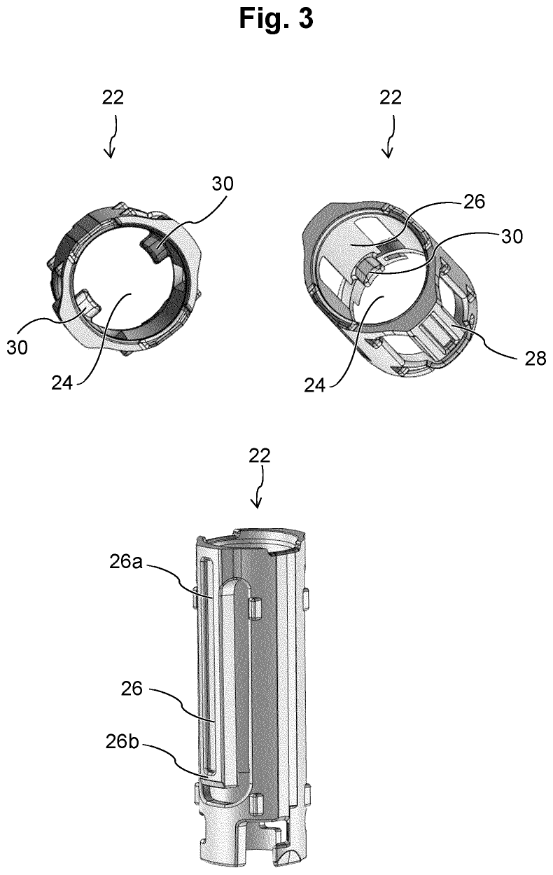

FIG. 3 shows a sleeve of a holder for an inhaler article;

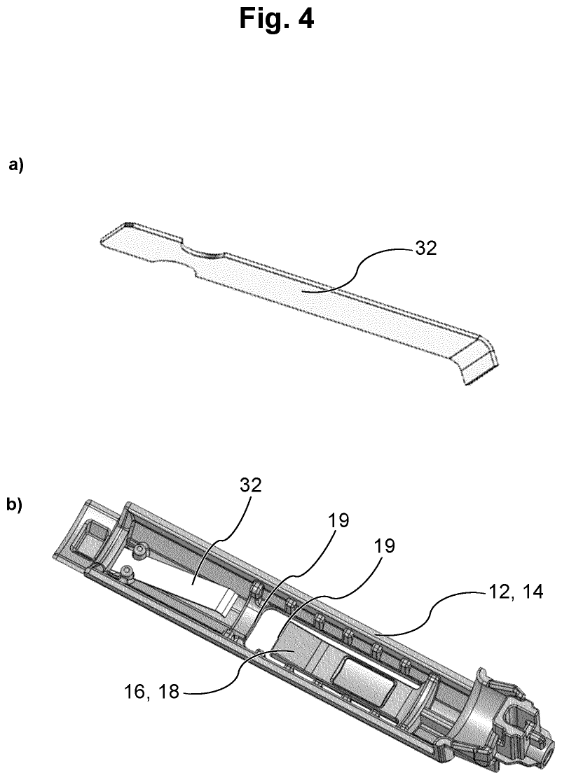

FIGS. 4a and 4b show a housing of a holder for an inhaler article;

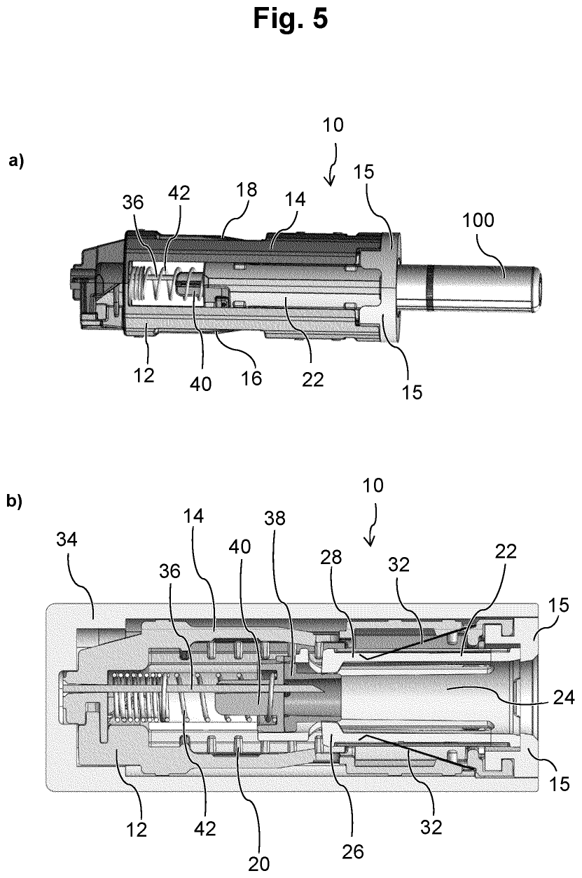

FIGS. 5a and 5b show a holder for an inhaler article; and

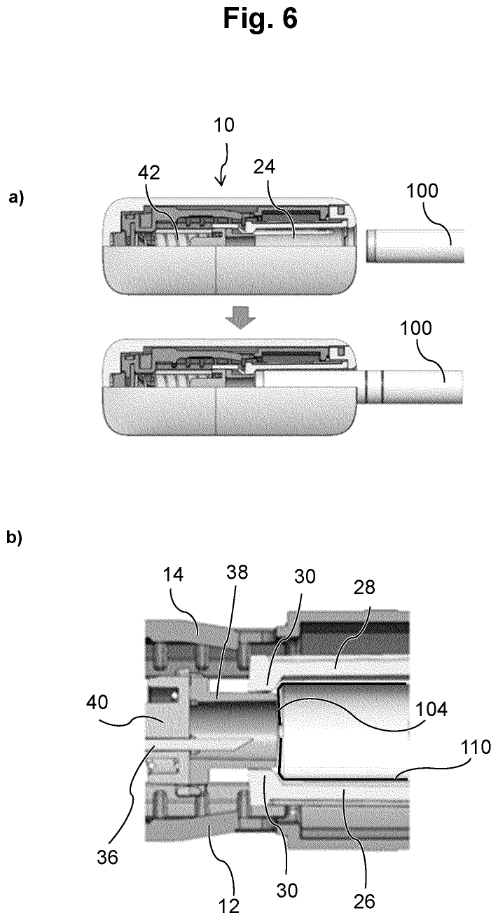

FIGS. 6 to 10 show a working principle of a holder for an inhaler article.

FIG. 1a shows an inhaler system including a holder 10 and a separate inhaler article 100. The holder 10 comprises an opening at a proximal end of the holder 10 for receiving at least a distal portion of the inhaler article 100. The inhaler article 100 is received within the holder 10 to activate or pierce a capsule disposed within the inhaler article 100. The inhaler article 100 may remain in the holder 10 during use by the user. The holder 10 and the inhaler article 100 may be configured to provide swirling inhalation airflow through the received inhaler article 100.

FIG. 1b shows an inhaler article 100 in cross-sectional view. The inhaler article 100 comprises a longitudinal center axis 102 extending between a distal end 104 and a proximal end 106. The distal end 104 is at least partly closed. The proximal end 106 is configured as a mouth-piece. The inhaler article 100 further comprises a capsule 108 arranged within a tubular element 110.

FIGS. 2a and 2b show a housing of a holder 10 for an inhaler article 100. The housing comprises a first housing main part 12 shown in two different perspectives in FIG. 2a. The housing comprises a second housing main part 14 shown in two different perspectives in FIG. 2b. The two main parts 12, 14 fit together forming one housing comprising a housing cavity, the housing cavity being surrounded by the connected two main parts 12, 14.

The housing comprises a retaining section for engaging with lever members of a sleeve. The retaining section comprises a first retaining element 16 of the first housing main part 12. The retaining section comprises a second retaining element 18 of the second housing main part 14. When the two main parts 12, 14 are connected to one another, the first and second retaining elements 16, 18 are located at opposite sides of the housing cavity.

An inner side of a wall of each of the first and second housing main parts 12, 14 comprises a plurality of circumferentially extending rib elements 20. The rib elements 20 may provide additional mechanical stability to the housing.

FIG. 3 shows a sleeve 22 of a holder 10 for an inhaler article 100 in three different perspectives. The sleeve 22 comprises a sleeve cavity 24 arranged to receive the inhaler article 100. The sleeve 22 comprises a first lever member 26 and a second lever member 28. The first and second lever members 26, 28 form part of a lockout mechanism of the holder 10. The first and second lever members 26, 28 are arranged at opposing sides of the sleeve cavity 24. The first and second lever members 26, 28 are integrally formed with a tubular body of the sleeve 22. Each of the first and second lever members 26, 28 comprises a first end (see first end 26a of the first lever member 26) coupled to, and integrally formed with, the tubular body of the sleeve 22, and a second end (see second end 26b of the first lever member 26) comprising a tip engagement feature 30 extending from the respective lever member 26, 28.

In the shown neutral configuration, the first and second lever members 26, 28 extend into the sleeve cavity 24. In more detail, the tip engagement features 30 of lever members 26, 28 extend into the sleeve cavity 24. The tip engagement features 30 are configured to directly contact the inhaler article 100 and to be moved by the contact with the inhaler article 100 when the inhaler article 100 is being inserted into the sleeve 22.

FIG. 4a shows a blade-shaped spring element 32 for pressing a lever member 26, 28 towards the neutral configuration.

FIG. 4b shows the spring element 32 being fixed to the housing. In particular, in the embodiment shown, a first end 32a of the spring element 32 is fixed to one of the first and second housing main parts 12, 14. A second end 32b of the spring element 32 is pointing into the housing cavity to press onto a respective lever member 26, 28 of the sleeve 22, when the sleeve 22 is positioned within the housing cavity.

The retaining section is shaped to hold at least a portion of each of the first and second lever members 26, 28 within the retaining section to resist movement of the sleeve 22 when the first and second lever members 26, 28 reach the retaining section. The retaining section comprises at least one abutment surface 19 arranged to resist lateral movement of a respective lever member 26, 28 when the lever member is in the working configuration and is engaged with the retaining section.

FIGS. 5a and 5b show a holder 10 for an inhaler article 100. FIG. 5a shows the holder 10 in side view. FIG. 5b shows the holder 10 in cross-sectional view. An inhaler article 100 is shown only in FIG. 5a. The holder 10 comprises a housing, the housing comprising a housing cavity. The housing comprises first and second housing main parts 12, 14 provided with circumferentially extending rib elements 20. The housing further comprises proximal housing parts 15. The proximal housing parts 15 may form a retaining ring element fixed to the open proximal end of the housing. The retaining ring element may retain the sleeve 22 within the housing cavity. The housing further comprises an outer shell 34 of the housing which is only shown in FIG. 5b and which is omitted in FIG. 5a.

The holder 10 comprises a sleeve 22 positioned within the housing cavity. The sleeve 22 comprises a sleeve cavity 24 arranged to receive the inhaler article 100 via an opening of the cavity 24 at the proximal end of the holder 10. The sleeve 22 is movable within the housing cavity between a first position and a second position. The holder 10 comprises a piercing element 36, preferably a needle, arranged to pierce the inhaler article 100 received within the sleeve 22 when the sleeve 22 is moved from the first position to the second position. The holder 10 comprises a lockout mechanism comprising a first lever member 26 of the sleeve 22, a second lever member 28 of the sleeve 22, and a retaining section of the housing.

The retaining section comprises a first retaining element 16 for engaging with the first lever member 26 and a second retaining element 18 for engaging with the second lever member 28. The first and second lever members 26, 28 are arranged at opposing sides of the sleeve cavity 24.

The holder 10 comprises a first spring element 32 for pressing the first lever member 26 towards the neutral configuration, and a second spring element 32 for pressing the second lever member 28 towards the neutral configuration. In FIG. 5b, the inhaler article 100 is not inserted and the sleeve 22 is in the first position with the first and second lever members 26, 28 being in the neutral configuration.

Each a first end of the first and second spring elements 32 is fixed to a respective housing main part 12, 14, and each a second end of the first and second spring elements 32 is pointing into the housing cavity to press onto the respective first and second lever members 26, 28 of the sleeve 22 positioned within the housing cavity.

The holder 10 comprises a tubular protruding element 38 arranged at a bottom end of the housing cavity. An outer diameter of the protruding element may be less than an inner diameter of the sleeve. The protruding element 38 is arranged to protrude into a distal end of the inhaler article on insertion of the inhaler article into the sleeve cavity. The protruding element 38 may be arranged to protrude into the sleeve cavity 24 when the sleeve 22 is in the second position. The protruding element 38 is connected to a connector element 40 which, in turn, connects to a helical spring 42. The helical spring 42 may push the sleeve 22 towards the first position. Thereby, the sleeve 22 may be automatically pushed from the second position into the first position when the first and second lever members 26,28 are in the neutral configuration. Due to inner channels of the protruding element 38 and the connector element 40, the piercing element 36 may be pushed through the protruding element 38 and the connector element 40.

FIGS. 6 to 10 show a working principle of a holder for an inhaler article, for example the holder of FIGS. 5a and 5b. FIGS. 6a to 10a show different configurations of the holder and the inhaler article 100 in side view, with an upper half of the holder being shown in cross-sectional view to show the interior components. FIGS. 6b to 10b show magnified cross-sectional views of a portion of the holder in the respective configuration. Arrows indicate a movement of components.

FIGS. 6a and 6b show a first step of the working mechanism of the holder 10.

FIG. 6a shows, at the top, the holder 10 and a separate inhaler article 100. FIG. 6a shows, at the bottom, the holder 10 after initial insertion of the inhaler article 100 into the sleeve cavity 24.

FIG. 6b shows a magnified cross-sectional view of a portion of the holder 10 in the configuration shown at the bottom of FIG. 6a with initially inserted inhaler article 100. The sleeve 22 is still in the first position. The first and second lever members 26, 28 are still in the neutral configuration. the helical spring 42 is in a relaxed state.

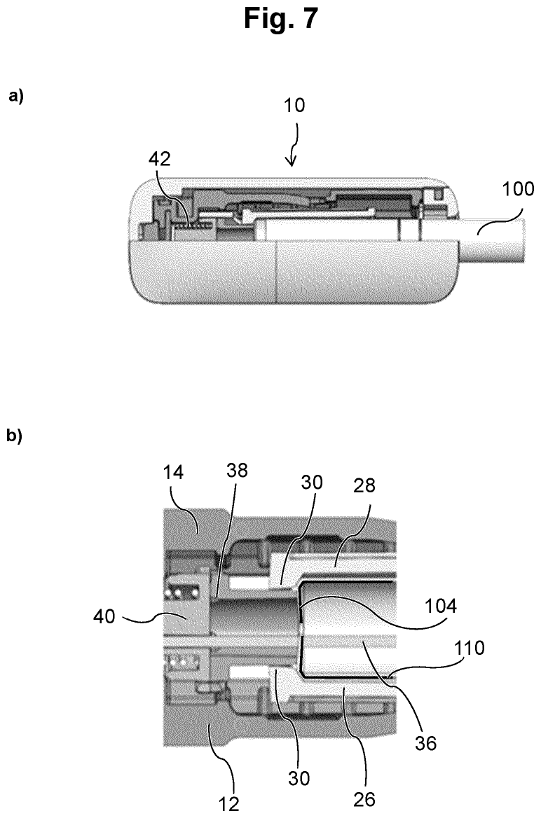

FIGS. 7a and 7b show a subsequent second step of the working mechanism of the holder 10. Upon further insertion of the inhaler article 100, the sleeve 22 has moved in a distal direction towards the piercing element 36 and into the second position. The sleeve 22 moves between the first and second positions along a longitudinal direction of the holder 10.

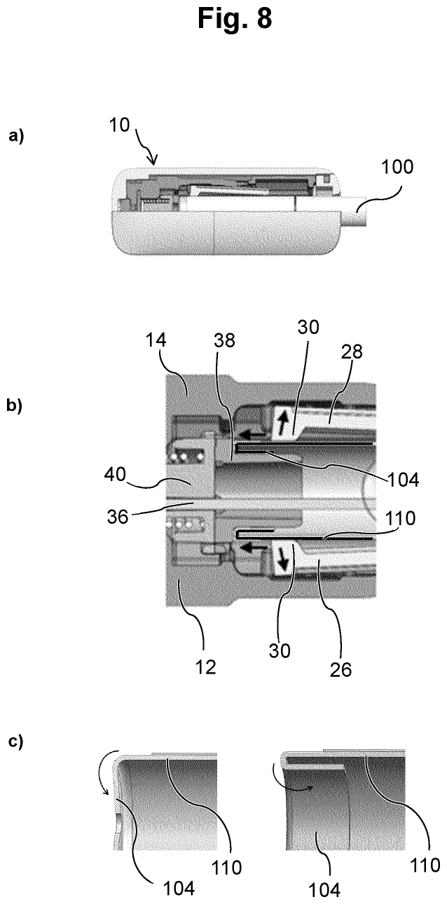

The protruding element 38 and the connector element 40 have also been pushed and moved into the distal direction by the further insertion of the inhaler article 100. As a result, the helical spring 42 is in a compressed state. Depending on the length of the piercing element 36 and the position of the capsule 108 within the inhaler article 100, the piercing element 36 may pierce a hole into a capsule 108 received in the inhaler article 100 in the step of FIGS. 7a and 7b. Alternatively, the piercing element 36 may pierce a hole into a capsule 108 received in the inhaler article 100 only after further insertion of the inhaler article 100 during a subsequent step as shown in FIGS. 8a to 8c.

FIGS. 8a to 8c show a subsequent third step of the working mechanism of the holder 10. The inhaler article 100 has been further inserted in a distal direction. Thereby, the distal end 104 of the inhaler article 100 has been deformed by the protruding element 38. Part of the distal end 104 has been flipped inwards. The distal end wall 104 of the inhaler article 100 has thus folded inwardly towards a tubular sidewall of the tubular element 110 upon the further movement of the inhaler article 100 with respect to the tubular protruding element 38. The protruding element 38 is arranged between the tip engagement features 30 of the first and second lever members 26, 28. The tubular folded distal end portion of the inhaler article 100 is coaxially arranged between the protruding element 38 and the tip engagement features 30. The folded end of the inhaler article 100 is shown in more detail in FIG. 8c. As shown in FIGS. 8a and 8b, the folded wall of the distal end 104 results in a thicker overall tubular sidewall of the inhaler article 100 at the distal part of the article 100, thereby pushing the first and second lever members 26, 28 in a radially outward direction from the neutral configuration into the working configuration. The holder 10 is arranged such that at least a portion of each the first and second lever members 26, 28 moves along a transverse direction when changing between the working configuration and the neutral configuration. The transverse direction is substantially perpendicular to the longitudinal direction of the holder.

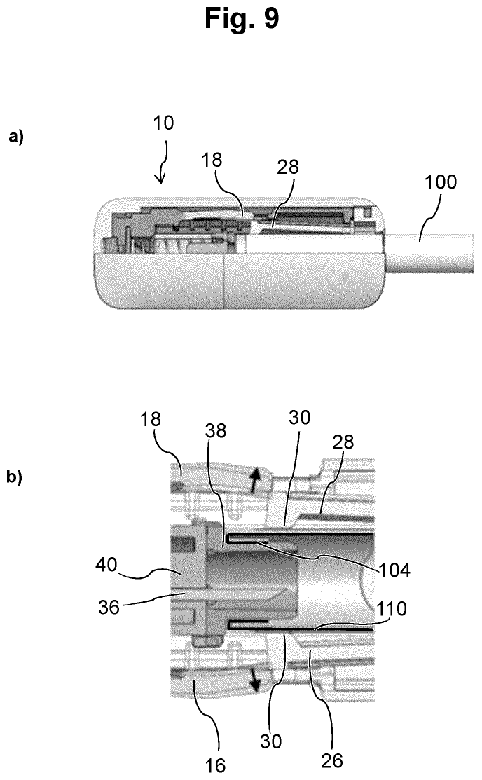

FIGS. 9a and 9b show a subsequent fourth step of the working mechanism of the holder. After piercing the hole into the capsule, the inhaler article 100 is released and moved into a proximal direction away from the piercing element 36. The first and second lever members 26, 28 are in the working configuration such that the first and second retaining elements 16, 18 are slightly bent into a radially outwards direction as the first and second lever members 26, 28 are moving by the first and second retaining elements 16, 18.

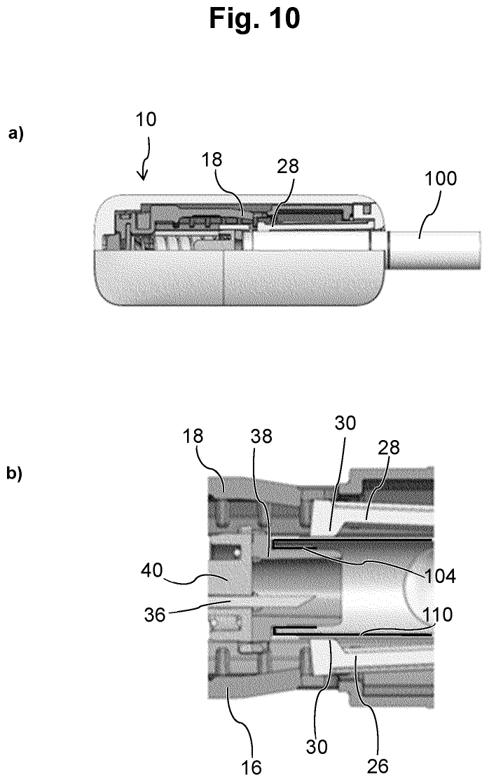

FIGS. 10a and 10b show a subsequent fifth step of the working mechanism of the holder. The sleeve 22 with the first and second lever members 26, 28 has been moved further in the proximal direction, and the first and second lever members 26, 28 have been moved to pass by the first and second retaining elements 16, 18. Now, the inhaler article 100 cannot move into a distal direction towards the piercing element 36 because the first and second lever members 26, 28, being pushed outwards into the working configuration are engaged with the first and second retaining elements 16, 18. The first and second retaining elements 16, 18 thus prevent movement of the sleeve 22. Thus, a second piercing of the capsule is not possible.

In a final sixth step, the inhaler article 100 has been completely removed from the holder 10. In absence of the inhaler article 100, the first and second lever members 26, 28 have moved back from the working configuration into the relaxed neutral configuration such that they disengage from the first and second retaining elements 16, 18. Consequently, the longitudinal movement of the sleeve 22 is no longer blocked by the retaining section. In turn, the helical spring 42 pushes the sleeve 22 back into the initial first position as shown in FIGS. 6a and 6b.

1.-17. (canceled)

18. A holder for an inhaler article, the holder comprising:
a housing comprising a housing cavity;
a sleeve positioned within the housing cavity, wherein the sleeve comprises a sleeve cavity configured to receive the inhaler article, the sleeve being movable within the housing cavity between a first position and a second position;
a piercing element configured to pierce the inhaler article received within the sleeve when the sleeve is moved from the first position to the second position;
a lockout mechanism comprising a first lever member of the sleeve, a second lever member of the sleeve, and a retaining section of the housing; and
a protruding element disposed at a bottom end of the housing cavity, the protruding element being configured to protrude into a distal end of the inhaler article on insertion of the inhaler article into the sleeve cavity,
wherein the first and the second lever members are configured to be in a neutral configuration when the inhaler article is not inserted into the sleeve,
wherein the first and the second lever members are further configured to be moved, by the inhaler article, from the neutral configuration into a working configuration when the inhaler article is being inserted into the sleeve,
wherein, when being in the working configuration, the first and the second lever members are further configured to engage with the retaining section as the sleeve moves from the second position back to the first position,
wherein the retaining section is arranged to hold the first and the second lever members when being engaged with the retaining section, thus retaining the sleeve in the first position, and
wherein the first and the second lever members are further configured to disengage from the retaining section when returning into the neutral configuration when the inhaler article is removed from the sleeve.

19. The holder according to claim 18, further comprising a first spring element configured to press the first lever member towards the neutral configuration, and a second spring element for pressing the second lever member towards the neutral configuration.

20. The holder according to claim 19, wherein the first and the second spring elements are blade-shaped.

21. The holder according to claim 19, wherein the first and the second spring elements are fixed to the housing.

22. The holder according to claim 21,
wherein each of a first end of the first and the second spring elements is fixed to the housing, and
wherein each of a second end of the first and the second spring elements is pointing into the housing cavity to press onto the respective first and the second lever members of the sleeve positioned within the housing cavity.

23. The holder according to claim 18, wherein the sleeve further comprises a polymeric material and a fiber material.

24. The holder according to claim 18, wherein the holder is configured such that:
the sleeve moves between the first and the second positions along a longitudinal direction of the holder, and
at least a portion of each the first and the second lever members moves along a transverse direction when changing between the working configuration and the neutral configuration, the transverse direction being substantially perpendicular to the longitudinal direction.

25. The holder according to claim 18, wherein the first and the second lever members extend into the sleeve cavity in the neutral configuration.

26. The holder according to claim 18, wherein the retaining section is shaped to hold at least a portion of each of the first and the second lever members within the retaining section to resist movement of the sleeve when the first and the second lever members reach the retaining section.

27. The holder according to claim 26, wherein the retaining section comprises at least one abutment surface arranged to resist lateral movement of the first and the second lever members.

28. The holder according to claim 18, wherein each of the first and the second lever members comprises a first end coupled to a tubular body of the sleeve and a second end comprising a tip engagement feature extending from the respective lever member.

29. The holder according to claim 28, wherein the tip engagement feature is configured to directly contact the inhaler article and to be moved by the contact with the inhaler article when the inhaler article is being inserted into the sleeve.

30. The holder according to claim 18, wherein the retaining section comprises a first retaining element configured to engage with the first lever member and a second retaining element configured to engage with the second lever member.

31. The holder according to claim 18, wherein the first and the second lever members are arranged at opposing sides of the sleeve cavity.

32. The holder according to claim 18, wherein the protruding element has a tubular shape.

33. The holder according to claim 18, wherein the protruding element is further configured to move together with the sleeve when the sleeve moves between the first and the second positions.

34. The holder according to claim 18, wherein a distal end of the piercing element is fixed to a distal end adjacent to or at a distal end of the housing.

35. An inhaler system comprising the holder according to claim 18 and an inhaler article, the inhaler article comprising a capsule disposed within a capsule cavity of the inhaler article.
